SEQUENCE LISTING (5 sequences)

SEQ ID NO: 1 - DNA, 47 bp, Artificial
  synthetic oligodeoxynucleotide
gagctgtttctgaggagccttcaagagaggctcctcagaaacagctc

SEQ ID NO: 2 - DNA, 99 bp, Homo sapiens
  misc_feature: promotor for human gene for H1 RNA
atatttgcatgtcgctatgtgttctgggaaatcaccataaacgtgaaatgtctttggatttgggaatcttataagttctgtatgagagcacagataggg

SEQ ID NO: 3 - DNA, 20 bp, Artificial
  synthetic oligodeoxynucleotide
gggagtccagttttctggac

SEQ ID NO: 4 - DNA, 24 bp, Artificial
  synthetic oligodeoxynucleotide
tggaaagtccagttttctggactt

SEQ ID NO: 5 - DNA, 20 bp, Artificial
  synthetic oligodeoxynucleotide
aggggtccagttttctggac

(19) United States
(12) Patent Application Publication
(10) Pub. No.: US 2009/0004703 A1
(43) Pub. Date: Jan. 1, 2009

(54) Method for the Production of Suitable Dna Constructs for Specific Inhibition of Gene Expression by Rna Interference

(75) Inventor: Matthias Schroff, Beriln (DE)

Correspondence Address: JOYCE VON NATZMER;PEQUIGNOT + MYERS LLC, 200 Madison Avenue, Suite 1901, New York, NY, 10016, US

(73) Assignee: MOLOGEN AG, Berlin (DE)

(21) Appl. No.: 11/569,697

(22) PCT Filed: Dec. 28, 2004
(86) PCT No.: PCT/DE04/02838

§ 371 Date: Dec. 15, 2006

(30) Foreign Application Priority Data
May 28, 2004 (DE) 10 2004 026 734.0

(51) Int. Cl. C12P 19/34; C12N 15/00

(52) U.S. Cl. 435/91.4; 435/320.1

(57) ABSTRACT

The invention relates to a method for the production of vectors which, following transfection thereof in eukaryotic cells, are suitable for targeted inhibition of the formation of defined proteins therein by RNA interference. The method for the production of such vectors does not include any PCR steps. It is a three-step procedure in a single reaction vessel and can be carried out within a few hours. Thus, a method is provided which allows very easy testing of a wide variety of siRNA sequences for their functionality within a very short time. Screening processes utilizing the rapid and uncomplicated production of vectors with the aid of said kit can be performed in a cost- and time-saving manner. Another advantage of vectors thus produced is their small size which, among other things, facilitates transfection.

The invention relates to a method for the production of vectors which, following transfection thereof in eukaryotic cells, are suitable for targeted inhibition of formation of defined proteins therein by RNA interference.

One recently detected way of inhibiting gene expression is based on the production of double-stranded RNA molecules. Using such double-stranded RNA (dsRNA), targeted switching off of single genes is possible in a highly effective manner and more rapidly compared to any other method, without impeding protein formation of neighboring genes. The basic principle is referred to as RNA interference, abbreviated as RNAi, and the dsRNA sequence responsible for this phenomenon as siRNA (small interference RNA).

The siRNA does not prevent reading of the gene, but rather switches on a cellular mechanism causing degradation of the mRNAs read from the gene, thus pre-venting formation of the corresponding protein (post-transcriptional gene silencing).

Such targeted mRNA degradation is triggered by short siRNA molecules 19-23 RNA bases in length which are homologous to the target mRNA whose trans-formation into a protein is to be prevented. The siRNA molecules combine with specific endoribonucleases to form a cellular RNA protein complex referred to as RISC (RNA-induced silencing complex). During formation of these complexes, the two RNA strands undergo dissociation, thereby forming so-called activated RISCs, each one including a single strand of the siRNA molecule. Activated RISCs including the antisense strand which is complementary to the target mRNA bind thereto, and the endoribonuclease of the RNA-protein complex subsequently provides for sequence-specific mRNA degradation.

The siRNA can be generated in the cell by way of experiment or can be incorporated by introduction from the outside. On the one hand, this can be done via synthetically produced siRNA molecules which can be administered both in vitro and in vivo.

However, this method has technical limitations. In addition to the general instability of synthetic siRNA both in a medium and in a cell, inhibition by means of a synthetic siRNA is, in principle, only possible in a transient fashion, and transfection of a large number of cells (e.g. neuronal cells) is extremely inefficient. For this reason, studies based on the transfection with synthetic siRNA are generally restricted in time to 1-5 days and in terms of a specific cell type. Furthermore, the high production cost and long production time are disadvantageous.

On the other hand, siRNA can be produced in the cell by vectors, said vectors being viral or plasmid-based vectors leading to formation of siRNA sequences later inside the cell by expression. The advantages over transfection with synthetic siRNA lie in a more stable and optionally regulated transcription of the corresponding siRNA sequence.

However, in addition to low transfection efficiency, plasmid-based vectors involve a complex production process. Thus, selection of stable clones is necessary, for example. During this process, usually being a lengthy one, which can take weeks or even months, a number of potential problems inherent to cloning experiments frequently arise. Checking the products requires sequencing which likewise is labor- and cost-intensive.

Further, plasmid-based vectors include antibiotic resistance genes required for the selection thereof. For this reason, such vectors are not suitable for use in living organisms. As a consequence of possible recombination with bacteria ubiquitous in the organism, there is a risk of increasing occurrence of antibiotic-resistant bacteria. Spreading of antibiotic resistances represents a serious problem and is unjustifiable.

Viral vectors are capable of efficient and targeted transfection, for which reason they offer advantages compared to synthetic siRNA molecules and plasmid-based vectors.

However, such viral vectors can be used in therapeutic applications only with reservations. Recombination of viral sequences with naturally occurring viruses represents an inherent safety risk in this case as well because it must be feared that new, pathogenic hybrid viruses would be formed. Moreover, the production thereof is also complex and cost-intensive.

Another way of producing vectors for siRNAs has been demonstrated by the Ambion Company on their internet site. The illustrated process avoids the above-mentioned drawbacks. Likewise, however, this production process is time-consuming and quite imperfect as a result of a number of necessary amplification steps of the respective sequences by means of PCR (polymerase chain reaction). It is very well possible that both undesirable and unnoticed mutations are produced which are even potentiated by the PCR process. In this case as well, control sequencings are required which prolong the production process and contribute to increased cost.

In view of the above prior art, the object of the invention is to provide a suitable method for the in vitro or in vivo synthesis of a defined siRNA sequence and a kit appropriate for this purpose.

Said object is accomplished by the characterizing features of claims 1 and 11.

In the meaning of the invention, siRNA sequence is understood to be the RNA sequence that is read from the DNA construct produced according to the invention. Hence, it is a singular RNA single strand being partially self-complementary.

In the meaning of the present invention the designation siRNA molecule is used for an siRNA which is formed as a result of refolding and base pairing of a self-complementary siRNA sequence. Hence, an siRNA molecule is a double-stranded RNA molecule in which the pairing strands are linked on one side by a non-complementary single strand.

According to the invention, a method is provided which is characterized by the following steps:
a) mixing a DNA double strand which includes a singular copy 19-23 bases in length of a gene sequence, once in 5′-3′ direction and once in 3′-5′ direction, a sequence 8-12 bases in length of two single strands being arranged between each 5′-3′ and 3′-5′ orientation of the singular copy of the gene sequence, said single strands being selected such that opposite bases are by no means complementary to each other and the flanking double strand regions are thereby linked to each other by two DNA single strands, said DNA double strand having short protruding ends of single-stranded DNA at the ends thereof,
   with
   hairpin loop-shaped oligodeoxynucleotides having short protruding ends of single-stranded DNA at the ends thereof,
   and
   a promoter having short protruding ends of single-stranded DNA, the single-stranded 5′ end of the promoter being capable of pairing with one of the hairpin loop-shaped oligodeoxynucleotides, and the single-stranded 3′ end of the promoter being complementary to the single-stranded 5′ end of the DNA double strand,
   and
   a termination signal for RNA polymerases with short protruding ends of single-stranded DNA, the 5′ protrusion of the termination signal being capable of specific pairing with the 3′ end of the DNA double strand, and the 3′ protrusion of the termination signal being capable of specific pairing with a hairpin loop-shaped oligodeoxynucleotide,
b) subsequent ligation of the DNA fragments, and
c) final purification of the vectors produced.

In a preferred embodiment, a method is provided wherein the promoter is part of a bacterially amplifyable plasmid which, prior to mixing the components in the first process step 1a), is cut with a restriction endonuclease recognizing a restriction site flanking the promoter on the plasmid, which restriction site is not present on the molecule to be produced.

According to the invention, it is also envisaged that in case of using a promoter as part of a bacterially amplifyable plasmid, the ligation step is effected in the presence of the restriction endonuclease by means of which the promoter has been excised from the plasmid.

In one embodiment the step of final purification is preceded by digestion of the reaction mixture, using an exonuclease specific for 3′ or 5′ DNA ends only.

In the method according to the invention, the DNA double strand added to the mixture at the beginning may result from an annealing of a partially self-complementary oligodeoxynucleotide or of two complementary oligodeoxynucleotides. It is also possible to effect annealing later in the reaction mixture, so that merely single-stranded complementary oligodeoxynucleotides are added at the beginning of the method according to the invention.

In a preferred embodiment the sequence of the oligodeoxynucleotides is selected in such a way that the resulting hairpins have the recognition sequence for a restriction endonuclease in their double-stranded region.

The final purification of the vectors produced by means of the method according to the invention is preferably effected using either chromatography or gel electrophoresis.

If the promoter is employed in the production process of the invention as part of a bacterially amplifyable plasmid, the restriction endonuclease by means of which the promoter can be excised from the plasmid is an enzyme from the group of class II restriction endonucleases, preferably from the group of BbsI, BbvI, BbvlI, BpiI; BplI, BsaI, BsmAI, BsmBI, BsmFI, BspMI, Eam1104I, EarI, Eco31I, Esp3I, FokI, HgaI, SfaNI or isoschizomers thereof.

The present invention is also directed to a kit used to carry out the method according to the invention, said kit including at least one promoter, hairpin loop-shaped oligodeoxynucleotides, and enzymes. The enzymes are ligases, restriction endonucleases, restriction exonucleases, kinases and polymerases or selected combinations thereof in the form of an enzyme mix. In addition, depending on the particular embodiment, the kit may include means for performing the enzymatic reactions, as well as means for purifying the vectors produced. The promoter can be included in the kit as part of a plasmid from which it can be excised using a suitable restriction endonuclease.

The present invention is also directed to a vector which is produced according to the method of the invention and which is capped by hairpin loop-shaped oligodeoxynucleotides having arranged therebetween a promoter at the 5′ end and a termination signal at the 3′ end of a DNA double strand, said DNA double strand including a singular copy 19-23 bases in length of a gene sequence, once in 5′-3′ direction and once in 3′-5′ direction, a sequence 8-12 bases in length of two single strands being arranged between each 5′-3′ and 3′-5′ orientation of the singular copy of the gene sequence, said single strands being selected such that opposite bases are by no means complementary to each other and the flanking double strand regions are thereby linked to each other by two DNA single strands. These expression cassettes are also referred to as minimalistic siRNA expression cassettes (MISECs).

The present invention differs from the well-known prior art in that a rapid method is provided by means of which a vector is produced which is free of plasmid or viral components and results in expression of siRNA sequences. The method for the production of such vectors does not involve any PCR steps, it is a three-step procedure and can be carried out in a single reaction vessel within a few hours. Thus, a method is provided which allows very easy testing of a wide variety of siRNA sequences for their functionality within a very short time. Screening processes for suitable siRNA sequences, utilizing the rapid and uncomplicated production of vectors with the aid of said kit, can be performed in a cost- and time-saving manner. Another advantage of the vectors thus produced is their small size which, among other things, facilitates transfection.

The siRNA sequences are single-stranded, comprising one sense strand and one antisense strand, each one comprising 19-23 nucleotides. The sense and antisense strands are separated by a short spacer region allowing subsequent folding of the strands to form a double-stranded siRNA molecule. This siRNA pairs with a target mRNA, resulting in degradation thereof by nucleases as described above.

The vector generated by means of said production process merely comprises a suitable promoter sequence, the siRNA sequence to be expressed, and a short termination sequence, and therefore does not bear any undesirable sequences of viral or plasmid origin. To protect from degradation by exonucleases, each end is covalently linked with a loop of single-stranded oligodinucleotides (ODN) so as to form a fully covalently capped molecule.

In an alternative production process according to the invention the DNA sequences complementary to each other, not separated by single-stranded regions, are located in a single vector. Each of the complementary double-stranded sequences 19-23 bases in length (sense and antisense) are included in separate vectors which can be produced in an analogous fashion using the method according to the invention. Consequently, the vectors thus produced have the same structure as the vector including the sense-loop-antisense DNA strand between promoter and hairpin loop, but lack the single-stranded region.

In principle, any eukaryotic promoter sequence such as the CMV promoter of cytomegalovirus is suitable as promoter for transcription control. It is preferred to use type III polymerase promoters such as H1 promoter, 7SK promoter, as well as the human and murine U6 promoter. The promoter sequence can be present on a suitable plasmid vector, from which it must be excised by means of restriction endonucleases at the beginning of production, but it is also possible to add the promoter sequence to the process in the form of a previously isolated or synthetically produced sequence.

Any known DNA sequence resulting in termination of expression via RNA polymerases is possible as termination sequence. Separate addition of the termination sequence to the process according to the invention is not necessary; instead, it can also be part of the double-stranded region of a hairpin loop-shaped oligodeoxynucleotide or of the 3′ end of the partial DNA double strand which has two single strands in the center thereof.

The siRNA sequence to be expressed is the sequence complementary to the target mRNA, which sequence is employed as PCR product in the production method according to the invention. Likewise, the siRNA sequence can be produced synthetically using oligodinucleotide synthesis. In this event, short ODN fragments can be used which must be annealed and ligated in a first step, but it is also possible to produce the entire siRNA sequence by means of ODN synthesis. The ODN fragment is phosphorylated by PN kinase.

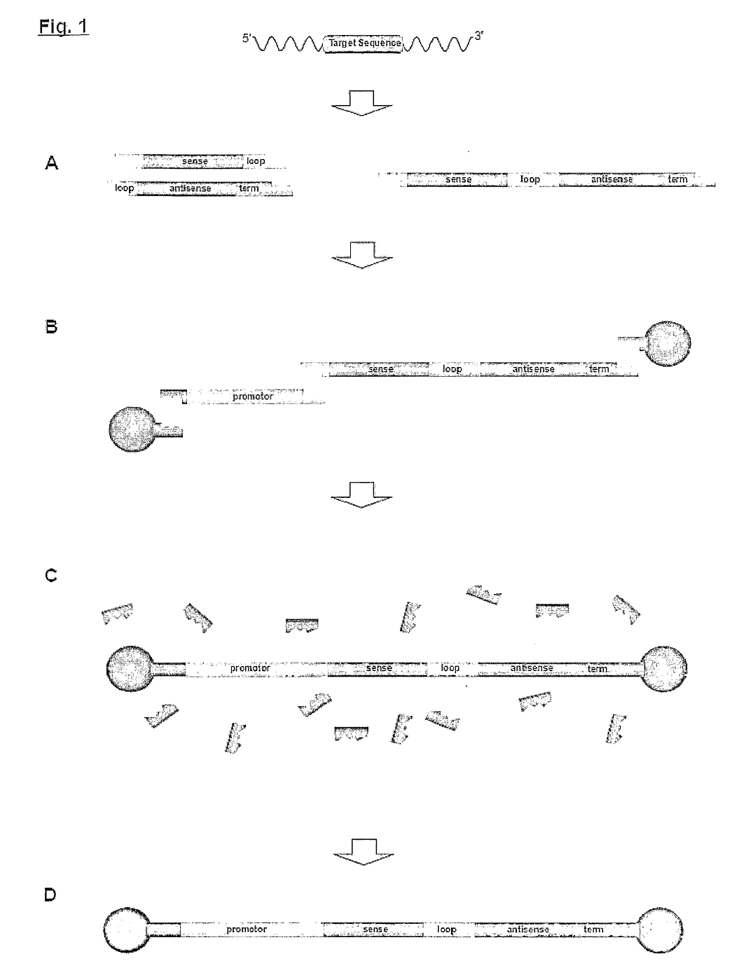

The production process of the method according to the invention can be described as follows and is illustrated in FIG. 1 for an overall view.

The plasmid bearing the promoter sequence is completely digested with the BspTNI restriction enzyme at 37° C. overnight, thereby providing the promoter fragment. Following addition of the respective siRNA sequence and 5′-phosphorylated hairpin loop-shaped oligodeoxynucleotides in double excess, the single fragments are ligated by means of the T4 DNA ligase enzyme in the presence of the BspTNI restriction enzyme. The resulting mixture of nucleic acids is treated with the T7 DNA polymerase enzyme. The final product, i.e., the vector expressing siRNA, is purified using column chromatography and is ready for transfection.

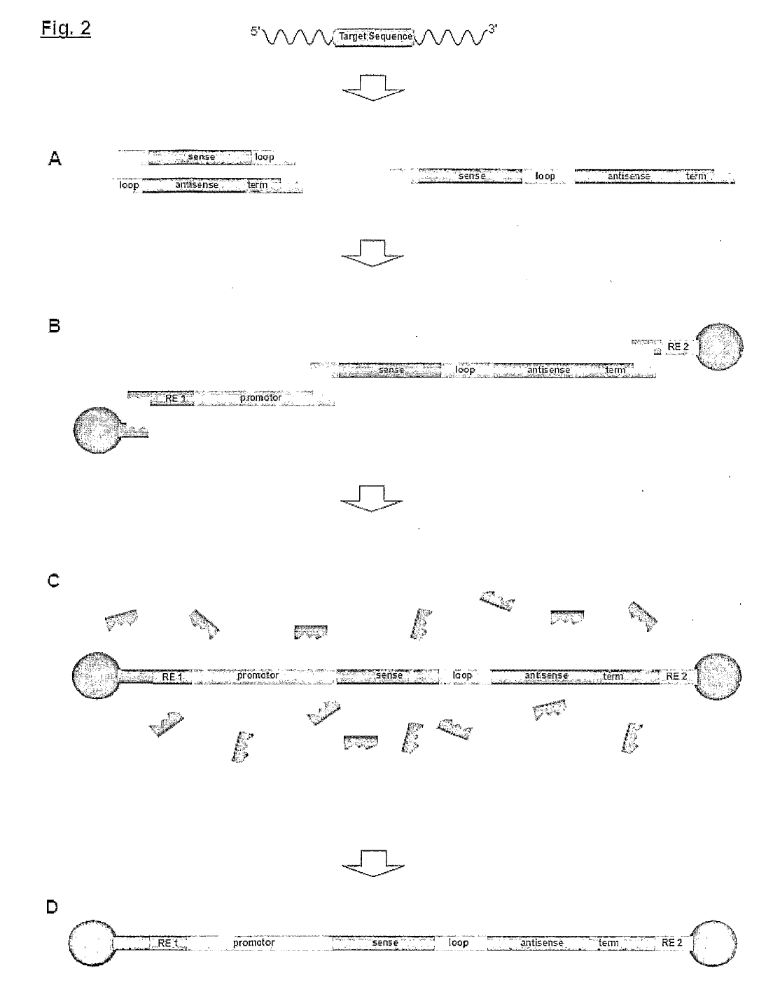

In one embodiment the vector expressing siRNA is envisaged to include two restriction sites allowing subsequent removal of the hairpins. This is advantageous in that the vector is available for further processes, be it cloning of the sequence into any desired expression vector, e.g. a plasmid, be it amplification of the sequence by PCR or the like. This embodiment is illustrated in FIG. 2.

An in vitro test was performed to check the functionality of the siRNA vectors according to the invention. To this end, hamster cells were transfected with various siRNA vectors intended to suppress the expression of luciferase. A plasmid and a vector according to the invention were used as siRNA vectors. Both vectors achieved about 90% inhibition of luciferase expression. While having comparable effectiveness and transfection efficiency, the method according to the invention advantageously achieves production of a vector which avoids the above-described disadvantages of plasmid-based vectors and can be produced in a much more time- and cost-saving fashion.

Hence, the significance of the invention lies in furnishing a method for the production of suitable vectors which can be used in screening procedures, thus serving in rapid functional testing of potential siRNA sequences. Furthermore, the kit provides a potential tool for gene therapy in a sense that pathologic genes are switched off.

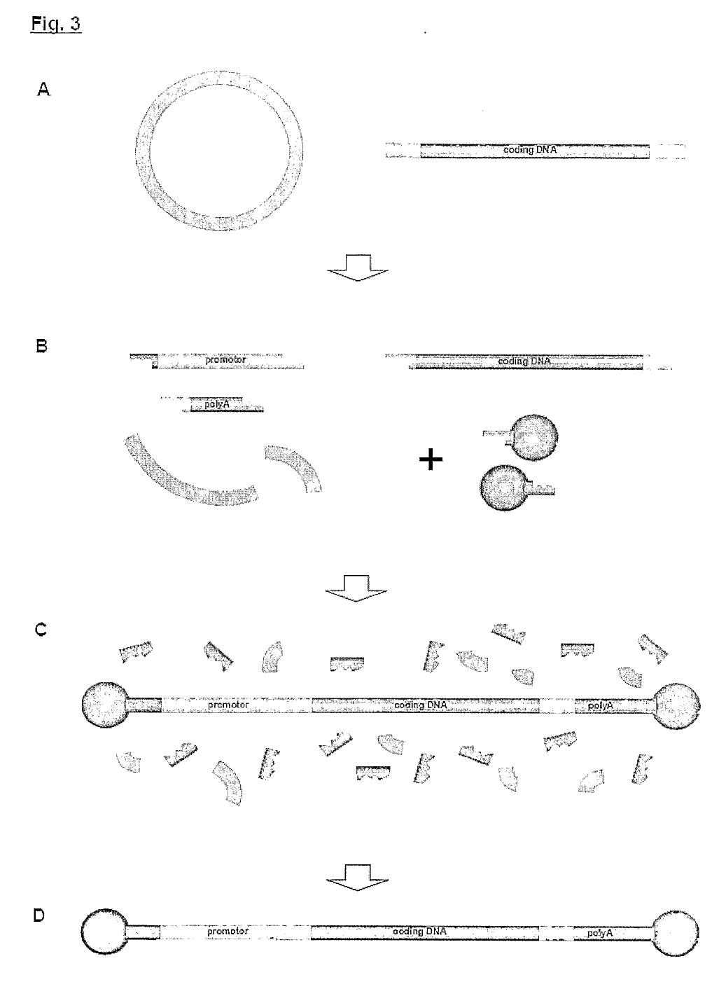

However, the production method also allows production of DNA expression vectors in a simple manner. To this end, the siRNA sequence is replaced by a DNA sequence encoding a gene. Using restriction digestion, unique protrusions are created at the ends of the DNA sequence, allowing ligation of the fragments (promoter, polyA site and hairpin loop-shaped ODN) in the proper arrangement. This pathway of production is schematically shown in FIG. 3. Also provided is a kit allowing the production of the DNA expression vectors. The components of the kits are the following: a suitable plasmid with promoter and polyA site sequences, coding DNA sequence, hairpin loop-shaped ODN, ATP, ligase, restriction enzyme, T7 polymerase, as well as column chromatography material for the purification of the product.

Further advantageous measures are described in the supplementary subclaims; the invention will be described in more detail with reference to the examples and the following figures.

FIG. 1 shows the production pathway of the siRNA vectors.
A: siRNA sequence to be employed in the process, which is homologous to the target mRNA. The sequence comprises a sense-antisense-loop region and a termination sequence. The siRNA sequence can be constituted of single ODN fragments which must be annealed, ligated and optionally phosphorylated by means of the enzyme mix, but it can also be present in the form of a complete ODN fragment.
B: shows the components ligated to the ODN fragment by the ligase enzyme. These components are the promoter sequence with corresponding complementary protrusions and the hairpin loop-shaped oligodinucleotides whose likewise complementary and unique protrusions of 4 bases each result in the formation of a covalently capped, linear vector which is constituted of the promoter, sense, loop, antisense and termination sequences and is capped at the ends in a hairpin loop shape.
C: Unligated components are degraded by T7 DNA polymerase digestion in a final step. The remaining product is purified using column chromatography.
D: Final product ready for transfection.

FIG. 2 shows the production pathway of the siRNA vectors with additional restriction sites.
A: siRNA sequence to be employed in the process, which is homologous to the target mRNA. The sequence comprises a sense-antisense-loop region and a termination sequence. The siRNA sequence can be constituted of single ODN fragments which must be annealed, ligated and optionally phosphorylated by means of the enzyme mix, but it can also be present in the form of a complete ODN fragment.
B: shows the components ligated to the ODN fragment by the ligase enzyme. These components are the promoter sequence with corresponding complementary protrusions, provided with an additional restriction site at the 5′ end, and the hairpin loop-shaped oligodinucleotides—a hairpin loop-shaped ODN likewise bearing an additional restriction site—whose likewise complementary and unique protrusions of 4 bases each result in the formation of a covalently capped, linear vector which is constituted of the promoter, sense, loop, antisense and termination sequences and is capped at the ends in a hairpin loop shape.
C: Unligated components are degraded by T7 DNA polymerase digestion in a final step. The remaining product is purified using column chromatography.
D: Final product ready for transfection, the product including two restriction sites.

FIG. 3 shows the production pathway of coding DNA vectors.
A: A plasmid and a coding DNA sequence are used as starting material. The plasmid bears restriction sites allowing excision of the promoter and polyA site sequences.
B: The following fragments are formed as a result of restriction digestion: promoter and polyA site, each having unique complementary protrusions, and residual plasmid fragments. The fragments are ligated after addition of coding DNA sequence, hairpin loop-shaped ODN and in the presence of ligase enzyme.
C: Unligated components are degraded using T7 DNA polymerase digestion. The covalently capped vector, constituted of promoter, coding and polyA site sequences, is purified by means of column chromatography.
D: DNA expressing vector usable in transfection.

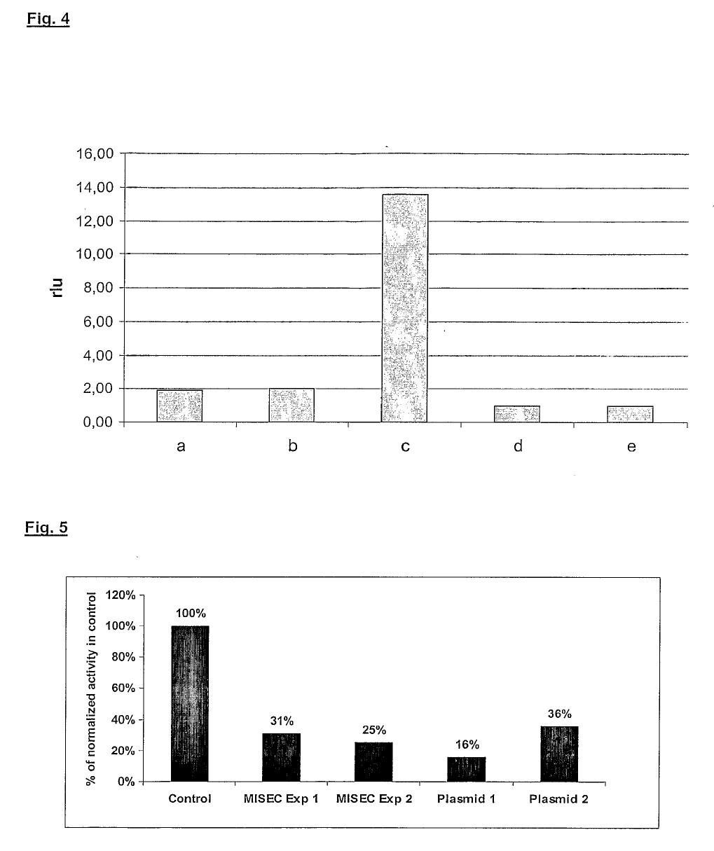

FIG. 4 shows the in vitro inhibition of luciferase expression by siRNA Expression was determined following transfection of CHOK1 cells, using relative light units (rlu). The following was used: siRNA vector produced using the method according to the invention (a), plasmid bearing siRNA sequence (b), positive control to control the luciferase expression (c), untreated cells (d), and cells transfected with empty vector (e). The values represent mean values calculated from a plurality of determinations. In the negative and positive controls, suppression of luciferase expression was not observed, as expected. In contrast, the siRNA-treated cells showed significantly lower luciferase expression. Luciferase expression is reduced by up to 90% compared to the positive control.

FIG. 5 shows the results of an experiment wherein the siRNA expression vector according to the invention is compared with plasmids containing identical expression cassettes.
CHO-K1 cells were cotransfected with 0.5 ng of plasmid encoding Renilla luciferase, 4.5 ng of plasmid encoding firefly luciferase, and 195 ng of the corresponding siRNA expression construct directed against the expression of firefly luciferase. The cells were lysed 24 hours after transfection, and the luciferase activity was determined in a luminometer. The activity of the firefly luciferase was balanced against the activity of Renilla luciferase and compared with the activity of the control (non-specific siRNA). The results illustrated show the mean values of three independent tests.
The suppression of gene expression by MISECs produced with an “siRNA Expression Vector Kit” using the method according to the invention is comparable to the effects of plasmid transfections, and the suppression of gene expression in both transfections ranges between 70 and 75%.

FIG. 6 shows the dose dependence of gene repression following transfection of MISECs produced with an “siRNA Expression Vector Kit”, in which case the hairpin siRNA was directed against the firefly luciferase gene, compared to non-specific siRNA sequences.
CHO-K1 were cotransfected with 500 ng of plasmid encoding Renilla luciferase, 100 ng of plasmid encoding firefly luciferase, and siRNA expression constructs against the firefly luciferase gene in the specified quantities. The cells were lysed after 24 hours, and the activity of the luciferases was determined in a luminometer. The activity of the firefly luciferase was balanced against the activity of Renilla luciferase and compared with the activity of the control (non-specific siRNA). The results illustrated show the mean values of two independent tests.
Compared to the non-specific siRNA, transfection of the same quantity (1000 ng) of MISECs produced with an “siRNA Expression Vector Kit” using the method according to the invention shows a significant decrease of the luciferase activity.

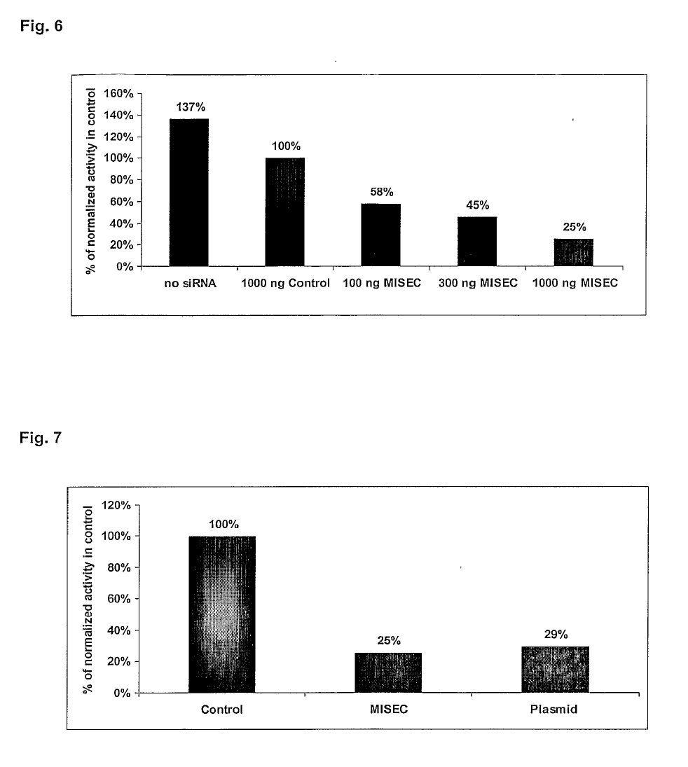

FIG. 7: In a further experiment carried out by the team of Dr. Christiane Kleuss at the Institut für Pharmakologie der Freien Universität Berlin, the effectiveness of gene repression by MISECs produced with an “siRNA Expression Vector Kit” using the method according to the invention was compared to that of a plasmid containing the same expression cassette.
CHO-K1 were cotransfected with 12 ng of plasmid encoding Renilla luciferase, 6 ng of plasmid encoding firefly luciferase, and 182 ng of a corresponding siRNA expression construct against the firefly luciferase gene. CHO-K1 were cotransfected with 500 ng of plasmid encoding Renilla luciferase, 100 ng of plasmid encoding firefly luciferase, and siRNA expression constructs against the firefly luciferase gene in the specified quantities.
The cells were lysed after 24 hours, and the activity of the luciferases was determined in a luminometer. The activity of the firefly luciferase was balanced against the activity of Renilla luciferase and compared with the activity of the control (non-specific siRNA). The results illustrated show the mean values of three independent tests.
In this experiment as well, the siRNA expression vectors produced according to the method of the invention show the same effectiveness as the plasmid with identical expression cassette, each time being about 75% reduction of the luciferase activity compared to the non-specific control plasmid. The inhibition of the constructs produced according to the invention is absolutely sufficient for effective identification of successful target sequences in a screening procedure within a short time, which are suitable for gene repression.

### EXAMPLE 1

### Production of siRNA Vectors for the Suppression of Luciferase Expression

The vectors encoding the siRNA of luciferase (siRNALuc) were obtained as follows:

The two ODN fragments for siRNALuc were heated at 90° C. for 3 min and annealed by slow cooling. In this way, the following sequence encoding luciferase was obtained:

SEQ ID NO. 1:
GAGCTGTTTC TGAGGAGCCT TCAAGAGAGG CTCCTCAGAA
ACAGCTC

Therein, the first 19 bases constitute the sense strand, the following 9 bases the loop region, and the remaining 19 bases the antisense strand.

Phosphorylation by means of PN kinase was effected subsequently. To obtain 10 micrograms of final product, an amount of 3.9 micrograms of siRNALuc was used. Following addition of 5.2 micrograms of H1 promoter (SEQ ID NO. 2) and of 5′-phosphorylated hairpin loop-shaped oligodeoxynucleotides (SEQ ID NO. 3 and 4):

SEQ ID NO. 2:
ATATTTGCAT GTCGCTATGT GTTCTGGGAA ATCACCATAA
ACGTGAAATG TCTTTGGATT TGGGAATCTT ATAAGTTCTGT
ATGAGAGCAC AGATAGGG
SEQ ID NO. 3:
5′-PH-GGG AGT CCA GTT TTC TGG AC-3′
(1.2 μg)
and
SEQ ID NO. 4:
5′-PH-TGG AAA GTC CAG TTT TCT GGA CTT-3′
(1.4 μg),

the individual fragments were ligated using the T4 DNA ligase enzyme in the presence of the BspTNI restriction enzyme. The resulting mixture of nucleic acids was treated with the enzyme T7 DNA polymerase. The final product, i.e., the vector expressing siRNALuc, was purified by column chromatography and was ready for transfection.

### EXAMPLE 2

### Suppression of Luciferase Expression In Vitro

Hamster cells of the CHOK1 cell line were seeded in 24-well plates in such a way that 8×10⁴ cells in 500 μl of medium were seeded per well. Following incubation over 24 h, transfection with various constructs expressing siRNALuc was effected. FuGene6 was used as transfection reagent. As reference vector for the determination of the firefly luciferase activity, a plasmid encoding Renilla luciferase was used in each batch in addition to plasmid encoding firefly luciferase. In this way, the firefly luciferase expression in relation to the marker Renilla luciferase expression can be determined by means of a dual assay. Non-transfected cells and cells transfected with a blank vector were used as negative controls. After incubation overnight, the cells were lysed and taken up in 15 μl of passive lysis buffer each time. Expression was detected using a dual luciferase reporter assay in a luminometer. The result is illustrated in FIG. 4.

### EXAMPLE 3

### Production of Coding DNA Vectors Using the Method According to the Invention

DNA vector production is effected in a combined restriction/ligation batch. The pMCV2.8 plasmid being used has four BspTNI and Eco31I restriction sites, providing promoter and polyA site after digestion.

The plasmid and the coding DNA fragment are digested with the BspTNI restriction enzyme and ligated with the hairpin loop-shaped ODNs. Sequences of the hairpin loop-shaped ODNs:

| | |
|---|---|
| SEQ ID NO. 3: | 5′-PH-GGG AGT CCA GTT TTC TGG AC-3′ |
| and | |
| SEQ ID NO. 5: | 5′-PH-AGG GGT CCA GTT TTC TGG AC-3′. |

Thus, the restriction/ligation batch includes: plasmid, coding DNA sequence, hairpin loop-shaped ODN, reaction buffer, ATP, BspTNI and T4 DNA ligase. Incubation is performed over 4 h at 37° C. The process is quenched by heat inactivation for 15 min at 70° C.

Degradation of residual vector and any non-ligated fragments is effected using T7 DNA polymerase digestion. The coding vector is purified by means of chromatography and is ready for transfection.

1. A method for producing vectors which, following transfection thereof into eukaryotic cells, specifically inhibit formation of defined proteins therein by RNA interference, said method comprising:
a) mixing
a DNA double strand which comprises
a singular copy, 19-23 bases in length, of a gene sequence, once in 5′-3′ direction and once in 3′-5′ direction,
a sequence, 8-12 bases in length, of two DNA single strands each being arranged between the 5′-3′ and 3′-5′ oriented singular copy of the gene sequence,
said single strands being selected such that opposite bases are by no means complementary to each other and double strand regions flanking them are linked to each other by said two DNA single strands, said DNA double strand having short protruding ends of single-stranded DNA at its ends,
with
hairpin loop-shaped oligodeoxynucleotides having short protruding ends of single-stranded DNA at their ends,
and
a promoter having short protruding ends of single-stranded DNA, a single-stranded 5′ end of the promoter being capable of pairing with one of the hairpin loop-shaped oligodeoxynucleotides, and a single-stranded 3′ end of the promoter being complementary to a single-stranded 5′end of the DNA double strand,
and
a termination signal for RNA polymerases with short protruding ends of single-stranded DNA, a 5′ protrusion of the termination signal being capable of specific pairing with a 3′ end of the DNA double strand, and a 3′ protrusion of the termination signal being capable of specific pairing with a hairpin loop-shaped oligodeoxynucleotide,
b) subsequent ligation of the DNA fragments, and
c) final purification of the vectors produced.

2. The method according to claim 1, wherein the promoter is part of a bacterially amplifyable plasmid which, prior to mixing the components in 1a), is cut with a restriction endonuclease recognizing a restriction site flanking the promoter on the plasmid, wherein the restriction site is not present on the molecule to be produced.

3. The method according to claim 2, wherein the ligation step according to 1 b) is effected in presence of the restriction endonuclease by means of which the promoter has been excised from the plasmid.

4. The method according to claim 2 or 3, wherein the step of final purification according to 1 c) is preceded by digestion of the reaction mixture, using an exonuclease specific for 3′ or 5′ DNA ends only.

5. The method according to at least one of claims 2 or 3, wherein the restriction endonuclease is an enzyme from the group of class II restriction endonucleases, preferably an enzyme from the group of BbsI, BbvI, BbvlI, BpiI; BplI, BsaI, BsmAI, BsmBI, BsmFI, BspMI, Eam104I, Earl, Eco31I, Esp3I, FokI, HgaI, SfaNI or isoschizomers thereof.

6. The method according to claim 1, wherein the mixture from 1 a) is added with a DNA double strand resulting from partial annealing of a partially self-complementary oligodeoxynucleotide or of at least two oligodeoxynucleotides.

7. The method according to claim 1, wherein the promoter being added is the promoter of the human gene for H1 RNA (SEQ ID NO. 2).

8. The method according to claim 1, wherein the hairpin loop-shaped oligodeoxynucleotides have a recognition sequence for a restriction endonuclease in their double-stranded region.

9. The method according to claim 1, wherein the purification in 1 c) is effected using chromatography and/or gel electrophoresis.

10. A kit comprising at least one promoter, hairpin loop-shaped oligodeoxynucleotides, and enzymes for production of vectors according to claim 1 which, following their transfection into eukaryotic cells, are suitable for targeted inhibiting formation of defined proteins therein by RNA interference.

11. The kit according to claim 10, wherein said enzymes are selected from restriction endonucleases, restriction exonucleases, ligases, kinases and polymerases.

12. The kit according to claim 10 or 11, wherein said kit additionally comprises means for-performing the enzymatic reactions.

13. The kit according to claim 10 or 11, wherein said kit additionally comprises means for purifying the vectors produced.

14. The kit according to claim 10, wherein the promoter is included as part of a bacterially amplifyable plasmid.

15. The kit according to claim 10, wherein said kit comprises a restriction endonuclease suitable for excision of the promoter from the plasmid.

16. A vector which, following transfection in eukaryotic cells, specifically inhibits formation of defined proteins by RNA interference, wherein said vector is capped by hairpin loop-shaped oligodeoxynucleotides having arranged therebetween a promoter at a 5′ end and a termination signal at a 3′ end of a DNA double strand, said DNA double strand comprising a singular copy, 19-23 bases in length, of a gene sequence, once in 5′-3′ direction and once in 3′-5′ direction, a sequence 8-12 bases in length of two single strands each being arranged between the 5′-3′ and 3′-5′ oriented singular copy of the gene sequence, said single strands being selected such that opposite bases are by no means complementary to each other and double strand regions flanking them are linked to each other by two DNA single strands.
